(19) United States
(12) Patent Application Publication
(10) Pub. No.: US 2026/0096789 A1
(43) Pub. Date: Apr. 9, 2026

(54) X-RAY DIAGNOSTIC APPARATUS

(71) Applicant: CANON MEDICAL SYSTEMS CORPORATION, Otawara-shi (JP)

(72) Inventors: Akitoshi SATO, Sakura (JP); Daisuke SATO, Utsunomiya (JP); Norimitsu KOSUGI, Nasushiobara (JP); Tsuyoshi OTSUKA, Nasushiobara (JP); Yoshimasa KOBAYASHI, Nasushiobara (JP)

(73) Assignee: CANON MEDICAL SYSTEMS CORPORATION, Otawara-shi (JP)

(21) Appl. No.: 19/347,024

(22) Filed: Oct. 1, 2025

(30) Foreign Application Priority Data
Oct. 3, 2024 (JP) 2024-174450
Aug. 25, 2025 (JP) 2025-139639

(51) Int. Cl. A61B 6/06; A61B 6/58

(52) U.S. Cl. CPC A61B 6/06; A61B 6/585

(57) ABSTRACT

An X-ray diagnostic apparatus according to the embodiment includes an X-ray tube, an X-ray diaphragm assembly, an X-ray detector, and processing circuitry. The X-ray diaphragm assembly includes an X-ray diaphragm defining an irradiation field of X-rays emitted from the X-ray tube and has a characteristic object. The X-ray detector is configured to detect the X-rays emitted from the X-ray tube and passed through a subject. The processing circuitry is configured to calculate information indicating a relative positional relationship between the X-ray tube and the X-ray detector based on an image of the characteristic object depicted in an X-ray image being based on transmission data acquired by the X-ray detector.

### CROSS-REFERENCE TO RELATED APPLICATION

This application claims the benefit of priority of Japanese Patent Application No. 2024-174450, filed on Oct. 3, 2024 and Japanese Patent Application No. 2025-139639, filed on Aug. 25, 2025, the entire contents of all of which are incorporated herein by reference.

### FIELD

Embodiments disclosed in this specification and drawings relate to an X-ray diagnostic apparatus.

### BACKGROUND

There are various types of X-ray diagnostic apparatuses such as general X-ray imaging apparatuses, mobile X-ray apparatuses for round, and X-ray TV apparatuses. Of these, the general X-ray imaging apparatuses and the mobile X-ray apparatuses have relatively simple configurations and perform X-ray imaging of, for example, the chest. The X-ray TV apparatuses can acquire X-ray images as still images. In addition, the X-ray TV apparatuses can also be configured to acquire moving X-ray images to perform intravascular treatment using medical devices such as catheters, i.e., IVR (Interventional Radiology).

In X-ray diagnosis using these X-ray diagnostic apparatuses, radiation exposure management is important. One method of radiation exposure management is called the NDD method (Numerical Dose Determination). The NDD method is a method for calculating the patient's surface radiation exposure from the imaging parameters during X-ray examination. One of the imaging parameters used in the NDD method is the source-to-image distance (SID). The SID is the distance from the focal point of the X-ray source to the X-ray detector. The SID is required to calculate the patient's surface radiation exposure based on the NDD method.

One method for obtaining the SID is to measure the distance from the focal point of the X-ray source to the X-ray detector using an external sensor such as an encoder. However, if this method is to be applied to a ceiling-suspended X-ray imaging device used in general X-ray imaging, it would be necessary to install a sensor such as an encoder and a belt on the ceiling rail to detect the position of the X-ray source. In this case, implementing these mechanisms would require considerable cost.

In the mobile X-ray apparatuses for round and the general X-ray imaging apparatuses, it is possible to manually measure the distance from the focal point of the X-ray source to the X-ray detector using a tape measure, etc. However, in clinical settings, it is time-consuming to manually measure the SID value using a tape measure, etc., and then input the measured SID value into the device.

On the other hand, there is also a method for obtaining the SID by referencing the SID value that is input in advance as an imaging protocol. However, this method does not allow for obtaining the SID value in the actual positioning state. Therefore, the calculated dose value may be inaccurate depending on the positioning state.

In addition, for example, in X-ray photography in a standing position or using the mobile X-ray apparatus for round, X-rays may not be incident on the X-ray detector from the front, but may be incident at an oblique angle to capture the image. In such cases, it would be very useful if information about the oblique angle could be measured by a simple method.

### BRIEF DESCRIPTION OF THE DRAWINGS

FIG. 1 is an external view showing an example of a configuration of an X-ray diagnostic apparatus 1 according to the first embodiment.

FIG. 2 is a diagram schematically showing a configuration of an X-ray tube assembly, an X-ray diaphragm assembly, and an X-ray detector, which are included in the configuration of the X-ray diagnostic apparatus of the first embodiment.

FIG. 3 is a block diagram showing an example of a functional configuration of the X-ray diagnostic apparatus 1 according to the first embodiment.

FIG. 4 is a diagram for explaining the first calculation method of the SID by the X-ray diagnostic apparatus 1 according to the first embodiment.

FIG. 5 is a diagram for explaining the second calculation method of the SID by the X-ray diagnostic apparatus 1 according to the first embodiment.

FIG. 6 is a diagram showing features, in which divisions 6a and 6b show the first modified example of the feature, and divisions 6c and 6d show the second modified example of the feature.

FIG. 7 is a diagram showing features, in which divisions 7a and 7b show the third modified example of the feature, and divisions 7c and 7d show the fourth modified example of the feature.

FIG. 8 is a diagram showing the fifth modified example of the feature.

FIG. 9 is a diagram showing the concept of a process for deleting the feature detected by the X-ray detector through image processing.

FIG. 10 is a block diagram showing a functional configuration of the second embodiment.

FIG. 11 is a diagram showing the concept of the oblique incidence angle calculation process in the first example of the second embodiment.

FIG. 12 is a diagram showing the concept of the oblique incidence angle calculation process in the second example of the second embodiment.

FIG. 13 is a diagram showing non-display processing as an alternative to image processing.

### DETAILED DESCRIPTION

Hereinafter, an embodiment of an X-ray diagnostic apparatus will be described in detail with reference to the drawings.

An X-ray diagnostic apparatus according to the embodiment includes an X-ray tube, an X-ray diaphragm assembly, an X-ray detector, and processing circuitry. The X-ray diaphragm assembly includes an X-ray diaphragm defining an irradiation field of X-rays emitted from the X-ray tube and has a characteristic object. The X-ray detector is configured to detect the X-rays emitted from the X-ray tube and passed through a subject. The processing circuitry is configured to calculate information indicating a relative positional relationship between the X-ray tube and the X-ray detector based on an image of the characteristic object depicted in an X-ray image being based on transmission data acquired by the X-ray detector.

### First Embodiment

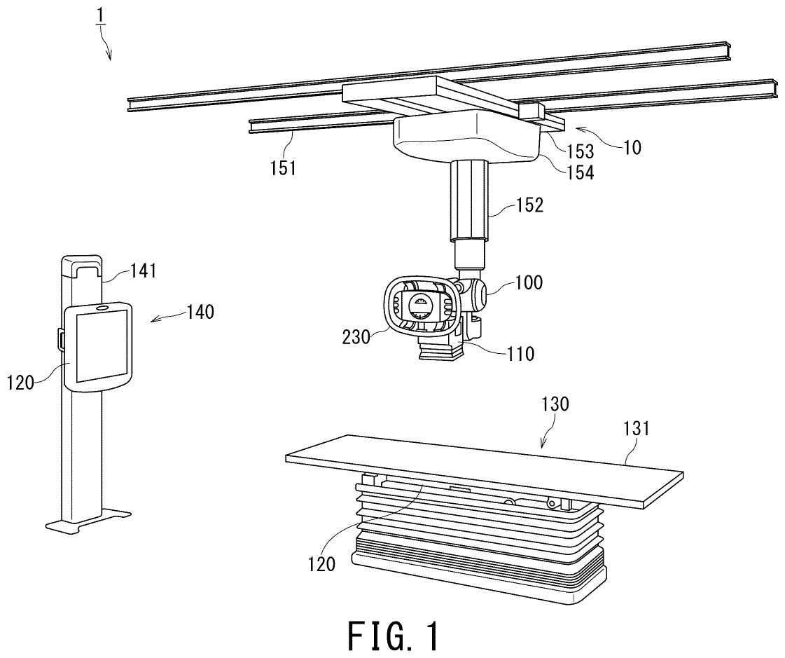

FIG. 1 is an external view showing an example configuration of an X-ray diagnostic apparatus 1 according to the first embodiment. The X-ray diagnostic apparatus 1 shown in FIG. 1 is a general X-ray imaging apparatus that performs simple X-ray imaging of the chest, etc. However, the X-ray diagnostic apparatus 1 is not limited to this and may be any apparatus that allows for the relative positional relationship between the X-ray detector and the X-ray focal point to be changed. For example, it may include a mobile X-ray apparatus for round that can be moved between hospital rooms or an X-ray TV apparatus. The X-ray diagnostic apparatus 1 includes, for example, an imaging device 10, a lying-posture imaging table 130, and an upright imaging table 140.

The imaging device 10 includes an X-ray tube assembly 100, an X-ray diaphragm assembly 110, and an X-ray detector 120. Of these, the X-ray tube assembly 100 and the X-ray diaphragm assembly 110 are held by an X-ray tube holding assembly 152. There are two types of X-ray tube holding assemblies 152, for example, a ceiling-type X-ray tube holding device and a floor-mounted X-ray tube holding device. FIG. 1 shows an example of the exterior of a ceiling-type X-ray tube holding assembly 152. The ceiling-type X-ray tube holding assembly 152 includes a ceiling rail 151, a cart unit 153, and an orthogonal movement assembly 154. The X-ray tube assembly 100 and the X-ray diaphragm assembly 110 are translated by moving the cart assembly 153 along the ceiling rail 151, and the X-ray tube assembly 100 and the X-ray diaphragm assembly 110 are translated by moving the orthogonal movement assembly 154 relative to the cart assembly 153 in a direction perpendicular to the extension direction of the ceiling rail 151. A user interface 230 (operation unit 230) is provided near the X-ray tube assembly 100 and the X-ray diaphragm assembly 110, and allows the angle and position of the X-ray tube assembly 100 and the X-ray diaphragm assembly 110 to be manually operated and the X-ray irradiation to be manually controlled.

The lying-posture imaging table 130 is configured as a bed on which a patient can be lying-posture on a table-top 131 for imaging. An X-ray detector 120, which is a component of the imaging device 10, is disposed below the table-top 131 of the lying-posture imaging table 130. On the other hand, the upright imaging table 140 is a device for imaging a subject P in an upright position. The upright imaging table 140 holds the X-ray detector 120 by a support stand 141 so that it can be moved up and down.

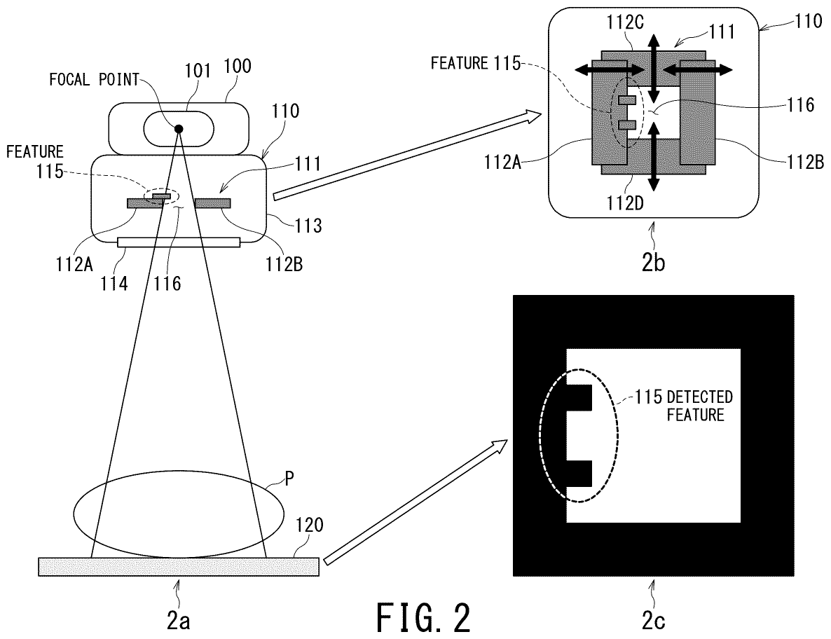

The X-ray detector 120 includes, for example, an FPD (Flat Panel Detector). X-rays emitted from an X-ray tube 101 (see FIG. 2) housed in the X-ray tube assembly 100 pass through a subject P (e.g., a patient P) on a table-top 131 and are detected by the X-ray detector 120.

FIG. 2 is a diagram schematically showing a configuration of the X-ray tube assembly 100, the X-ray diaphragm assembly 110, and the X-ray detector 120, which are included in the configuration of the X-ray diagnostic apparatus 1 of the first embodiment.

As shown in a division 2a of FIG. 2, the X-ray tube assembly 100 has an X-ray tube 101 built in, and X-rays are emitted from the X-ray tube 101 toward the subject P. In the division 2a of FIG. 2, the focal point of the emitted X-rays is indicated by a black circle.

The X-ray diaphragm assembly 110 includes an X-ray diaphragm 111, which is housed in a case 113. The X-ray diaphragm 111 is a device that regulates the position and size of the X-ray irradiation field relative to the subject P. As shown in a division 2b of FIG. 2, the X-ray diaphragm 111 is configured with, for example, four diaphragm blades 112A, 112B, 112C, and 112D. The diaphragm blades 112A, 112B, 112C, and 112D are made of a material that blocks X-rays, such as lead, iron, and tungsten. Hereinafter, the four diaphragm blades 112A, 112B, 112C, and 112D may be collectively referred to simply as diaphragm blades 112.

The four diaphragm blades 112A, 112B, 112C, and 112D are independently moved in four directions indicated by black arrows in the division 2b of FIG. 2. This makes it possible to control the size and position of an opening 116 formed by the diaphragm, which transmits the X-rays emitted from the X-ray tube 101. By controlling this X-ray diaphragm 111, it is possible to control the position and size of the X-ray irradiation field relative to the subject P.

A cover member 114 that is transmissive to X-rays and visible light is provided on the outer peripheral surface of the case of the X-ray diaphragm assembly 110, on the surface in the direction in which X-rays are emitted toward the subject P. The cover member 114 is formed of a resin material such as an acrylic plate.

In the X-ray diagnostic apparatus 1 according to the embodiment, the X-ray diaphragm assembly 110 is configured to include a “feature 115,” which is an example of a characteristic object having a required feature detectable by the X-ray detector 120. Like the diaphragm blades 112, the feature 115 is made of a material that blocks X-rays, such as lead, iron, and tungsten. The size of the feature 115 is determined in accordance with the SID, and the relative size of the feature 115 to the diaphragm blades 112 shown in FIG. 2 and subsequent figures are for convenience's sake. The size of the feature 115 includes the vertical length and horizontal length of the feature 115 when viewed from the X-ray tube 101 (or X-ray detector 120) side. The feature 115 can be provided at any position between the X-ray tube 101 and the X-ray detector 120. Specifically, the feature 115 can be provided on, for example, the X-ray diaphragm 111 or the cover member 114 of the X-ray diaphragm assembly 110.

In the first example of the feature 115 shown in FIG. 2, two characteristically shaped appendages (e.g., two convex protrusions) are provided on one of the four diaphragm blades 112 (e.g., diaphragm blade 112A). This feature 115 protrudes into a portion of the opening 116. Therefore, as shown in portion 2c of FIG. 2, the shape of the feature 115 is detected in a portion of the periphery of the radiation field of the X-ray detector 120.

The “feature 115” may be an addition having a characteristic shape attached to a part of the X-ray diaphragm 111 or the cover member 114, or may be a notch formed by cutting out a part of the X-ray diaphragm 111 in any characteristic shape (for example, a concave shape). The former addition is provided so that a part of the side surface of the diaphragm blade 112 extends toward the opposing diaphragm blade 112. The latter notch is provided so that a part of the side surface of the diaphragm blade 112 is recessed toward the opposing side surface.

The “feature 115” may be a through-hole of any characteristic shape formed in a part of the X-ray diaphragm 111. For example, the “feature 115” may be a through-hole formed in a part of the X-ray diaphragm 111 in a characteristic shape of a figure such as a circle, a square, or a triangle, or a characteristic shape imitating a symbol, a number, a letter, or the like.

The “feature 115” may be a pattern of any characteristic shape provided on the cover member 114, such as an acrylic plate. For example, the “feature 115” may be a grid pattern provided on the cover member 114, or a pattern of characteristic shapes such as circles, squares, triangles, symbols, numbers, letters, etc.

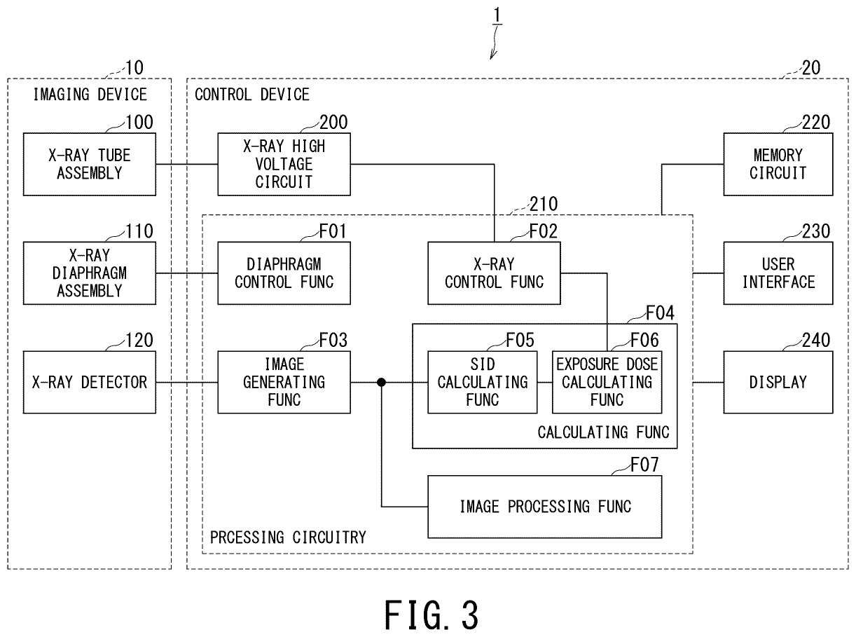

FIG. 3 is a block diagram showing an example of a functional configuration of the X-ray diagnostic apparatus 1 according to the first embodiment. The X-ray diagnostic apparatus 1 includes an imaging device 10 and a control device 20. As described above, the imaging device 10 includes an X-ray tube assembly 100, an X-ray diaphragm assembly 110, and an X-ray detector 120. On the other hand, the control device 20 includes an X-ray high voltage circuit 200, processing circuitry 210, a memory circuit 220, a user interface 230, and a display 240.

The X-ray high voltage circuit 200 applies a high voltage and a tube current to the X-ray tube under the control of the X-ray control function F02 of the processing circuitry 210.

The memory circuit 220 stores programs for the processor of the processing circuitry 210, as well as X-ray images and various data generated by the processing circuitry 210.

The user interface 230 includes an input device that can be operated by a user and an input circuit that inputs signals from the input device. The input device can be realized by, for example, an operation console, a joystick, a trackball mouse, a keyboard, a touch panel that performs input operations by touching the operation surface, a touch screen that combines a display screen and a touchpad, a non-contact input circuit that uses an optical sensor, an audio input circuit, or the like.

The display 240 is configured by a general display output device such as a liquid crystal display or an OLED (organic light emitting diode) display. The display 240 displays various data in addition to the X-ray images generated by the processing circuitry 210. Note that all or part of the X-ray images and data displayed on the display 240 may be displayed on a touch panel or touch screen of the user interface 230.

The processing circuitry 210 has one or more processors. The following functions are realized by software processing performed by executing programs stored in the memory circuit 220. Alternatively, the processing circuitry 210 may be configured to realize each function by hardware processing performed by an FPGA (Field Programmable Gate Array) or an ASIC (Application Specific Integrated Circuit), or may realize each function by a combination of software processing and hardware processing.

As shown in FIG. 3, the processing circuitry 210 realizes a diaphragm control function F01, an X-ray control function F02, an image generating function F03, a calculating function F04, and an image processing function F05. Of these, the calculating function F04 has an SID calculating function F05 and an exposure dose calculating function F06 as its internal components.

The diaphragm control function F01 controls the size and position of the opening 116 adjusted by the X-ray diaphragm 111 in accordance with control data input by the user via the user interface 230. The X-ray control function F02 also outputs a control signal to the X-ray high voltage circuit 200 to control the tube voltage and tube current of the X-ray tube in accordance with control data input by the user via the user interface 230 or control data defined in a preset protocol.

The image generating function F03 generates an X-ray image based on transmission data output from the X-ray detector 120. As described above, the X-ray diagnostic apparatus 1 according to the first embodiment is configured to provide the feature 115 in the X-ray diaphragm assembly 110. This feature 115 is detected by the X-ray detector 120, and the detected feature 115 is depicted in the X-ray image generated by the image generating function F03. The calculating function F04 calculates information indicating a relative positional relationship between the X-ray tube 101 and the X-ray detector 120 based on the image of the feature 115 depicted in the X-ray image based on the transmission data acquired by the X-ray detector 120.

The SID calculating function F05 of the calculating function F04 calculates information indicating the relative positional relationship between the X-ray tube 101 and the X-ray detector 120 as an X-ray imaging-related index. The calculation is based on at least the dimensions of the feature 115 detected by the X-ray detector 120, i.e., the dimensions of the feature 115 depicted in the X-ray image (hereinafter referred to as detected dimensions). An example of an X-ray imaging-related index is the SID. A specific method for calculating the SID will be described later.

The exposure dose calculating function F06 of the calculating function F04 calculates the exposure dose, such as the patient surface exposure dose, using the NDD method based on the SID calculated by the SID calculation function F05, as well as information such as the tube current and tube voltage input by the X-ray control function F02, etc.

The image processing function F07 performs various image processing on the X-ray images generated by the image generating function F03, as well as performing delete processing to make inconspicuous feature 115 that are not necessary for interpreting the X-ray images for diagnosis.

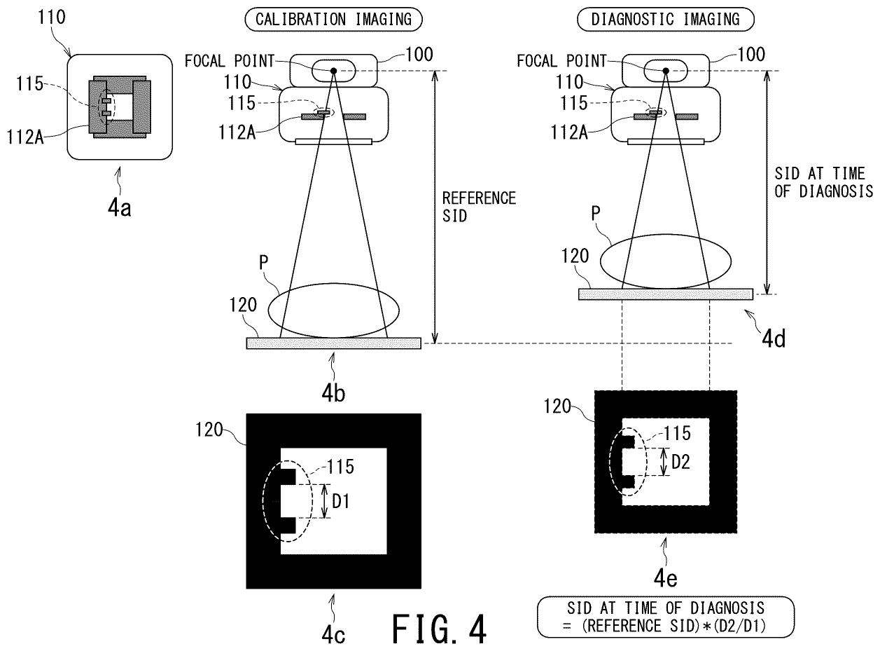

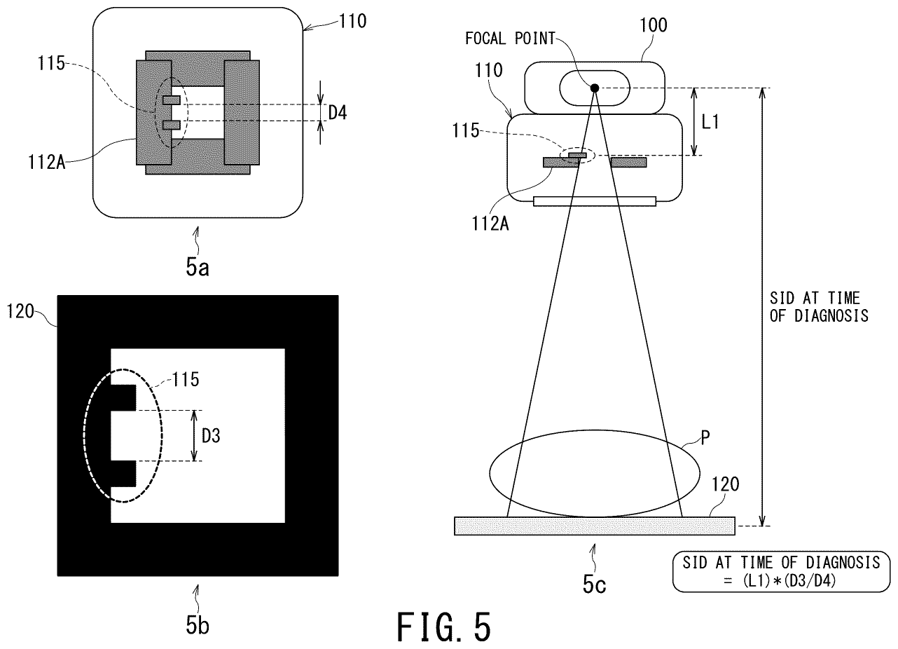

FIG. 4 is a diagram showing the first SID calculation method by the X-ray diagnostic apparatus 1 according to the first embodiment. FIG. 5 is a diagram showing the second SID calculation method. As described above, the type of feature 115 used to calculate the SID is not particularly limited. In FIGS. 4 and 5, an example will be described in which two convex protrusions provided on one diaphragm blade 112A are used as the feature 115, as shown in a division 4a of FIG. 4 and a division 5a of FIG. 5.

In the first SID calculation method, as shown in divisions 4b and 4c of FIG. 4, calibration imaging is performed separately from normal diagnostic imaging. The calibration imaging acquires the detected dimensions of the feature 115 detected by the X-ray detector 120. For example, the distance D1 between the opposing sides of two convex protrusions is acquired as the reference dimension D1 of the feature 115 in the calibration imaging (hereinafter referred to as the reference detected dimension). The SID in the calibration imaging is measured using an appropriate measuring device, such as a tape measure. Then, the measured SID is set as the reference SID in the processing circuitry 210, for example, via the user interface 230.

On the other hand, in normal diagnostic imaging, when the SID at the time of diagnosis is unknown, the detected dimension D2 of the feature 115 detected by the X-ray detector 120 (i.e., the distance D2 between the opposing sides of the two convex-shaped protrusions provided on the diaphragm blade 112A) is obtained, as in calibration imaging. Here, the SID at the time of diagnosis refers to the SID at the time of diagnostic imaging.

Then, the SID calculating function F05 of the processing circuitry 210 calculates a ratio (i.e., (D2/D1)) between the reference detection dimension D1 of the feature 115 and the detection dimension D2 of the feature detected by the X-ray detector 120 in diagnostic imaging when the SID is unknown, and calculates the unknown diagnostic SID using the reference SID, which is a known SID. Specifically, the diagnostic SID is calculated based on the following equation (1).

$$SID at the time of diagnosis = ( reference SID ) * ( D 2 / D 1 ) \qquad ( 1 )$$

Divisions 5a to 5c of FIG. 5 show the second SID calculation method. In the second SID calculation method, the SID is calculated using only normal diagnostic imaging without performing calibration imaging. However, in the second calculation method, the actual dimension D4 of the feature 115, as shown in the division 5a of FIG. 5, and the distance L1 from the X-ray focus to the feature 115, as shown in the division 5c of FIG. 5, are used to calculate the diagnostic SID. Here, the actual dimension D4 of the feature 115 is, for example, the distance D4 between the opposing sides of two convex protrusions provided on the diaphragm blade 112A, as in the division 4a of FIG. 4.

Furthermore, the distance L1 is the distance L1 from the X-ray focus to the two convex protrusions provided on the diaphragm blade 112A in the direction perpendicular to the X-ray focus toward the X-ray detector 120.

In diagnostic imaging, when the SID at the time of diagnosis is unknown, the detected dimension D3 of the feature 115 detected by the X-ray detector 120 (i.e., the distance D3 between the opposing sides of the two convex protrusions on the diaphragm blade 112A) is obtained, as shown in the division 5b of FIG. 5.

Then, the SID calculating function F05 of the processing circuitry 210 calculates a ratio (i.e., (D3/D4)) between the actual dimension D4 of the feature 115 and the detected dimension (i.e., D3) of the feature 115 detected by the X-ray detector 120 during diagnostic imaging when the SID is unknown, and calculates the unknown diagnostic SID using the distance L1 from the X-ray focus to the feature 115. Specifically, the diagnostic SID is calculated based on the following equation (2).

$$SID at the time of diagnosis = ( L 1 ) ⋆ ( D 3 / D 4 ) \qquad ( 2 )$$

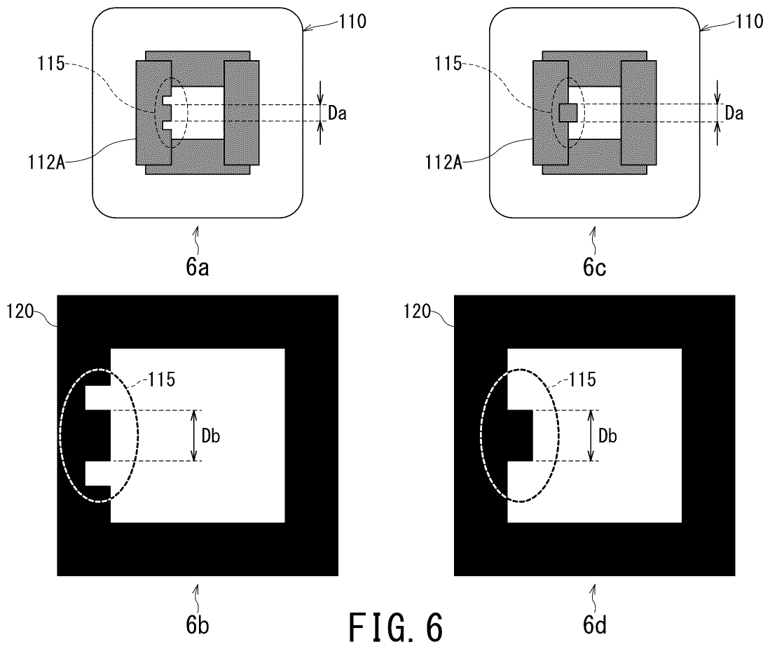

Next, with reference to FIGS. 6 to 8, several modified examples of the feature 115 provided on the X-ray diaphragm assembly 110 and the feature 115 detected by the X-ray detector 120 will be shown.

Divisions 6a and 6b of FIG. 6 are diagrams showing the first modified example of the feature 115. While the feature 115 of the first embodiment described above is a convex protrusion (convex portion) provided on the side surface of the diaphragm blade 112A, the feature 115 of the first modified example is a concave notch (concave portion) provided on the side surface of the diaphragm blade 112A. With this first modified example, too, the diagnostic SID can be calculated by applying the detected dimension Db between the two notches 115 detected by the X-ray detector 120 or the actual dimension Da between the two notches 115 to the equation (1) or (2).

Divisions 6c and 6d of FIG. 6 show the second modified example of the feature 115. The feature 115 in the first embodiment described above is multiple (specifically, two) convex protrusions provided on the diaphragm blade 112A. The diagnostic SID is calculated by applying the distances D1 and D2 or the distances D3 and D4 between the multiple (specifically, two) features 115 to the equation (1) or (2). In contrast, in the second modified example, a single characteristic shape provided on the diaphragm blade 112A is used as the feature 115, and the length of a predetermined portion of this feature 115 is used as the actual dimension of the feature 115 or the detected dimension detected by the X-ray detector 120. For example, as shown in the division 6c of FIG. 6, the length of one side of one protrusion provided on the diaphragm blade 112A is used as the actual dimension Da of the feature 115, and the length of the corresponding side detected by the X-ray detector 120 is used as the detected dimension Db. Then, by applying the actual dimension Da or the detected dimension Db to the equation (1) or (2), the diagnostic SID can be calculated.

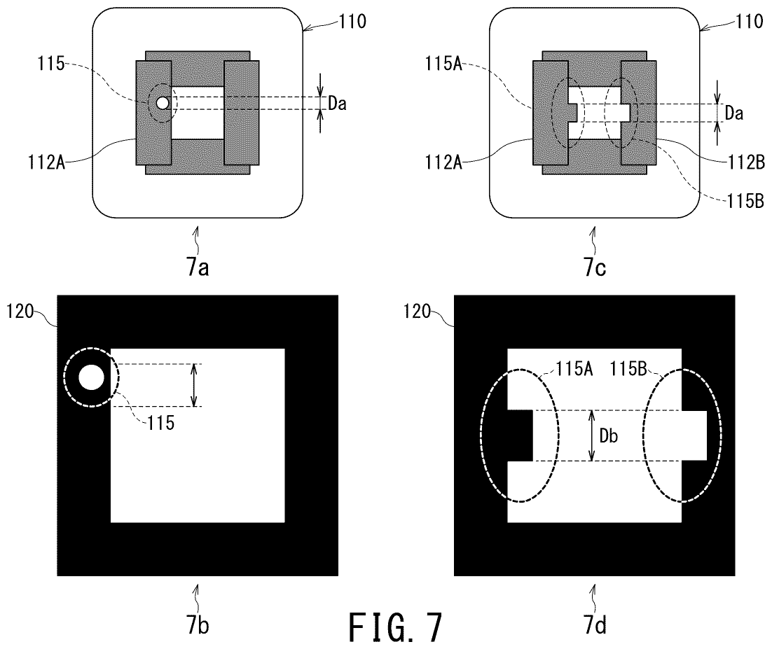

Divisions 7a and 7b of FIG. 7 show the third modified example of the feature 115. In the third modified example, a through-hole of any characteristic shape is provided in a predetermined diaphragm blade 112 (e.g., one diaphragm blade 112A), which is all or part of the X-ray diaphragm 111. That is, the feature 115 according to the third modified example is a portion of the predetermined diaphragm blade 112 that constitutes the X-ray diaphragm 111, in which a through-hole for passing X-rays is provided. The divisions 7a and 7b of FIG. 7 show a circular through-hole, but the through-hole may have a characteristic shape, such as a square or triangle, or a symbol, number, letter, or the like. The through-holes in the divisions 7a and 7b of FIG. 7 enable the SID to be acquired with a simple configuration without processing the side surface of the diaphragm blade 112 facing the opposing diaphragm blade 112.

In the divisions 7a and 7b of FIG. 7, the diaphragm blade 112A is configured to have one through-hole (i.e., the feature 115). However, the diaphragm blade 112A may be configured to have multiple (e.g., two) through-holes (i.e., the features 115). In this case, the SID can be calculated based on the distance between the multiple (e.g., two) through-holes.

Divisions 7c and 7d of FIG. 7 show the fourth modified example of the feature 115. The feature 115 of the fourth modified example is a convex feature 115A provided on the first diaphragm blade 112A, which is one of the multiple diaphragm blades constituting the X-ray diaphragm 111, and a concave feature 115B provided on a side surface of the second diaphragm blade 112B facing the first diaphragm blade. The convex feature 115A and the concave feature 115B are formed to fit together when the X-ray diaphragm 111 is closed. The through-holes in the divisions 7a and 7b of FIG. 7 allow the convex tip of the feature 115A to be inserted into the concave portion of the diaphragm blade 112B as the opening narrows. Therefore, the feature 115A does not abut against the side surface of the opposing diaphragm blade 112B, making it possible to acquire the SID with a simple configuration even when the opening is very small.

The feature 115 in the first embodiment is attached to the upper side (X-ray tube side) of the diaphragm blade 112A as an additional part separate from the diaphragm blade 112A, as shown in the divisions 2a and 2b in FIG. 2, for example. This is to prevent the diaphragm blade 112A and the diaphragm blade 112B from interfering with each other when the X-ray diaphragm 111 is closed. In contrast, in the fourth modified example of the feature 115, the shape of the feature 115 is formed in the X-ray diaphragm 111 itself so that they do not interfere with each other even when the X-ray diaphragm 111 is closed, and therefore no additional part is required.

Note that in the divisions 7c and 7d of FIG. 7, one convex feature 115A is provided on the diaphragm blade 112A, and one concave feature 115B is provided on the side surface of the diaphragm blade 112B. This is not a limitation, and a configuration may be adopted in which multiple (e.g., two) convex features 115A are provided on the diaphragm blade 112A, and multiple (e.g., two) concave features 115B are provided on the side surface of the diaphragm blade 112B. In this case, the SID can be calculated based on the distance between the multiple features 115A (or between the multiple features 115B).

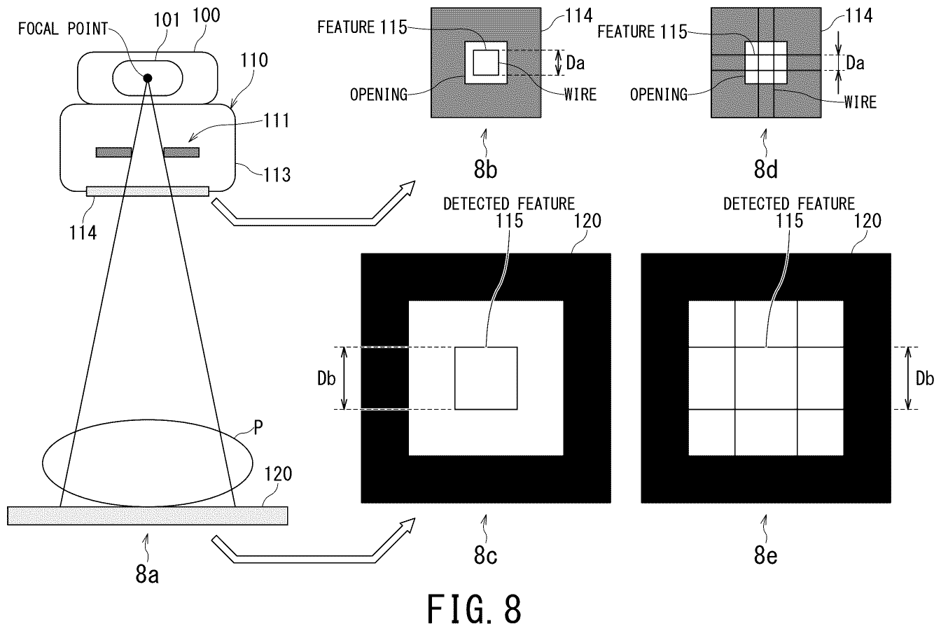

Divisions 8a to 8e of FIG. 8 are diagrams showing the fifth modified example of the feature 115. The feature 115 of the fifth modified example is a pattern provided on a cover member 114 (e.g., an acrylic plate) that is part of the case 113 that houses the X-ray diaphragm 111, and the cover member 114 is provided on a transmission window through which X-rays pass toward the subject. The shape of the pattern provided on the cover member 114 is not particularly limited, and a pattern of any shape may be used. For example, a rectangular pattern as shown in the divisions 8b and 8c of FIG. 8 may be used, or a grid pattern as shown in the divisions 8d and 8e of FIG. 8 may be used. The SID can then be calculated based on a dimension Da of a portion of the pattern provided on the cover member 114 and a detected dimension Db of the corresponding portion detected by the X-ray detector 120.

The method for forming the pattern on the cover member is not particularly limited. For example, the pattern can be formed by attaching a tungsten wire with a diameter of about 100 μm to the cover member 114.

When forming a grid-like pattern such as that shown in the divisions 8d and 8e of FIG. 8, the pattern can be formed using four wires. The captured image shows the four wires superimposed on the image of the subject. Since the wires attached to the cover member 114 are close to the focal point, they appear as blurred wire images. Therefore, this image is first subjected to a Hough transform. In the image after the Hough transform, the images of the four wires are expressed as dots. However, since the image after the Hough transform also contains image information other than the wires, threshold processing is used to replace low pixel values with zero. An inverse Hough transform is then performed to create an image in which only the wires are extracted. The SID can then be calculated by calculating the distance between the wires using threshold processing. When measuring the distance between the wires, the SID may be calculated using the average value in both the horizontal and vertical directions.

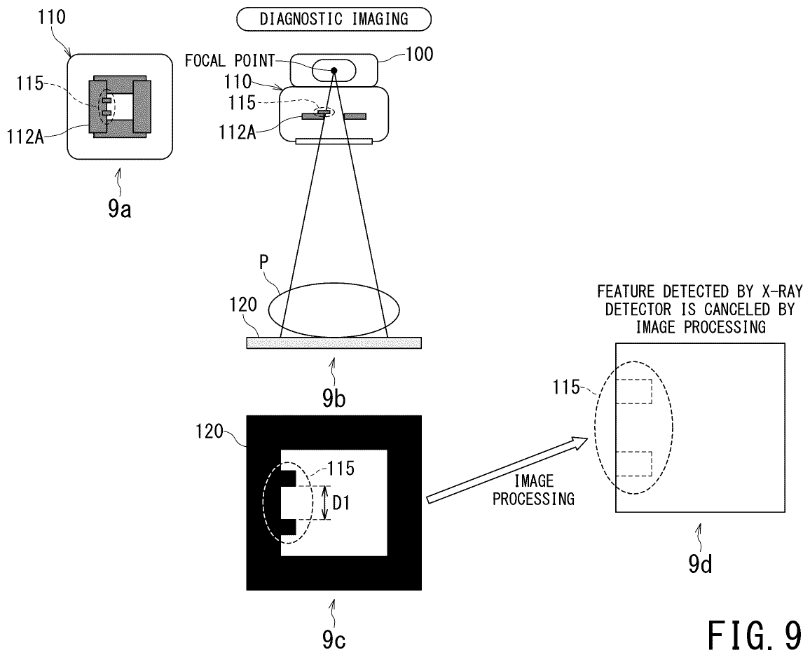

Divisions 9a to 9d of FIG. 9 are diagrams showing the concept of a process for deleting the region of the feature 115 detected by the X-ray detector 120 through image processing in order to make the feature 115 less noticeable on the X-ray image. The feature 115 of the first embodiment and the features 115 of each of the modifications described above are detected by the X-ray detector 120, and therefore would be depicted in the X-ray image if left as is. Therefore, depending on the size and position of the feature 115, it is possible that this may interfere with image diagnosis. Therefore, in the X-ray diagnostic apparatus 1 according to the first embodiment, the image processing function F07 of the processing circuitry 210 deletes the feature 115 detected by the X-ray detector 120 and depicted in the X-ray image through image processing, as shown in the divisions 9c and 9d of FIG. 9.

The process of deleting the features 115 depicted in the X-ray image by image processing can also be applied to the fifth modified example of the features 115 that form a pattern such as a grid on the cover member 114 using wires or the like.

### Second Embodiment

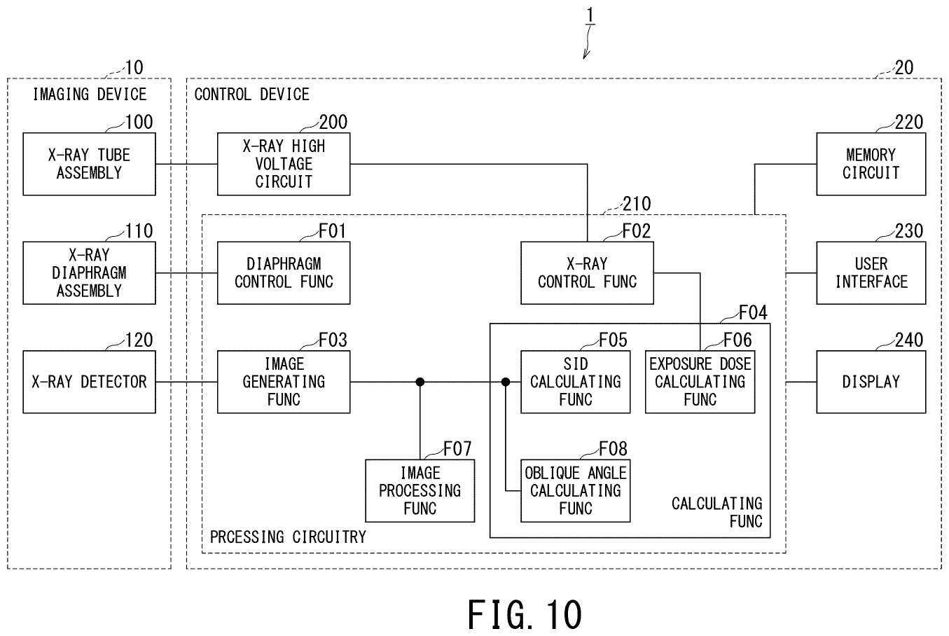

FIG. 10 is a block diagram showing a functional configuration according to the second embodiment. The second embodiment differs from the first embodiment in that the calculating function F04 realized by the processing circuitry 210 further includes an oblique angle calculating function F08, but the other configurations are the same as those of the first embodiment.

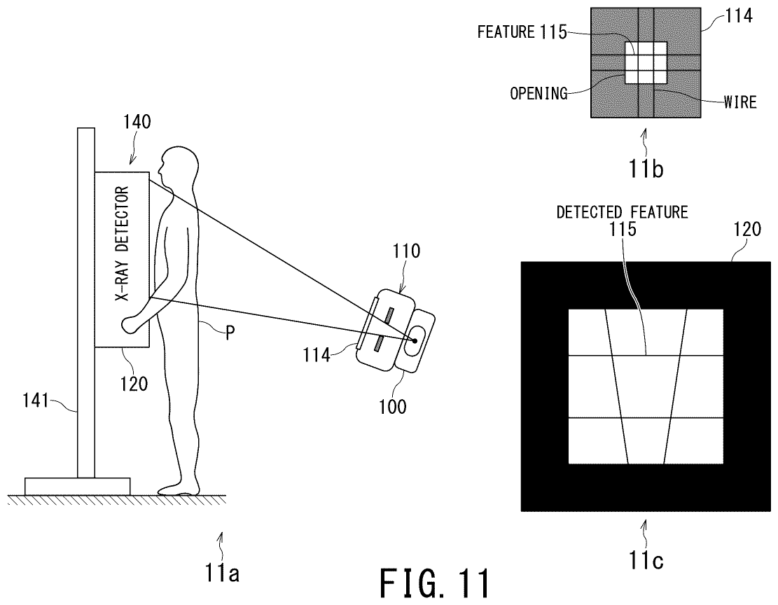

Divisions 11a to 11c of FIG. 11 illustrate the concept of the oblique incidence angle calculation process in the first example of the second embodiment. In normal X-ray imaging, the X-ray tube 101 and the X-ray detector 120 are directly opposed to each other. However, depending on the purpose of imaging, imaging may be performed with X-rays incident on the X-ray detector 120 at an oblique angle, as shown in the division 11a of FIG. 11. Furthermore, when using the mobile X-ray apparatus 1 to image a patient in a hospital room or a patient undergoing surgery, it may be difficult to image the X-ray tube 101 and the X-ray detector 120 directly opposed to each other. In such imaging, it is useful to know the incidence angle of the X-rays with respect to the X-ray detector 120, i.e., information on the oblique incidence angle.

In the first example of the second embodiment, similarly to the fifth modified example of the first embodiment, a grid-like pattern using wires or the like is used as the feature 115. An oblique angle calculating function F08 of the processing circuitry 210 calculates the oblique incidence angle of X-rays with respect to the X-ray detector 120 as an X-ray imaging-related index based on the degree of distortion of the grid-like pattern detected by the X-ray detector 120. The oblique incidence angle of X-rays is an example of an X-ray imaging-related index. The calculated oblique incidence angle is output to, for example, the user interface 230 and provided to the user.

Consider, for example, a situation in which X-rays are irradiated from below to above the subject P, as shown in the division 11a of FIG. 11. The feature 115 of a grid pattern that intersects with each other at right angles, as shown in the division 11b of FIG. 11, is detected by the X-ray detector 120 as a state in which the vertical wires are inclined at an angle, as shown in part 11c of FIG. 11. Detecting the inclination angle of this wire from the X-ray image allows the oblique incidence angle to be calculated.

Alternatively, the relationship between the wire tilt angle and the X-ray oblique incidence angle may be obtained in advance by experiment or the like, and this relationship may be stored in advance as, for example, a lookup table.

In this case, the oblique incidence angle associated with the wire tilt angle detected in diagnostic imaging is obtained by referring to the lookup table.

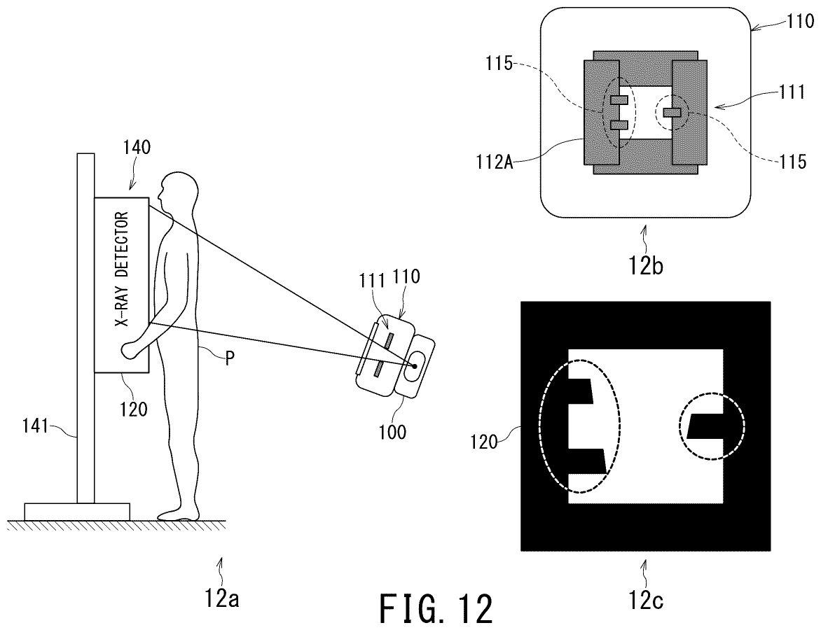

Divisions 12a to 12c of FIG. 12 are diagrams showing the concept of the oblique incidence angle calculation process in a second example of the second embodiment. The feature 115 used in this second example is a characteristic pattern including position information of at least three spatially separated points.

The oblique angle calculating function F08 of the processing circuitry 210 calculates the oblique incidence angle of X-rays with respect to the X-ray detector 120 as an X-ray imaging-related index. The calculation is based on the difference between the shape of a first characteristic pattern (the characteristic pattern including the position information of at least three spatially separated points) provided on the X-ray diaphragm assembly 110 and the shape of the second characteristic pattern detected by the X-ray detector 120. The calculated oblique incidence angle is output to, for example, the user interface 230 and provided to the user.

### Non-Display Processing as an Alternative to the Image Processing Mentioned Above

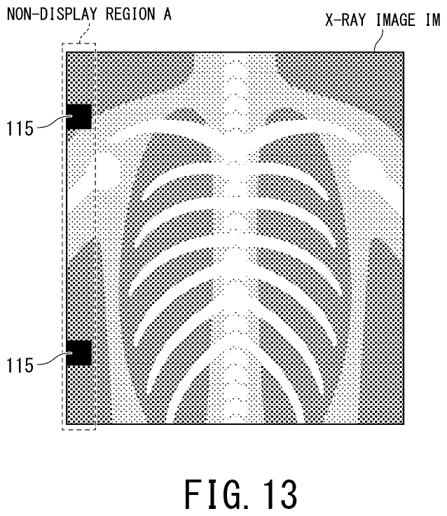

The method of processing in which the image processing function F07 deletes the region of the feature 115 detected by the X-ray detector 120 through image processing has been described using the divisions 9a to 9d in the FIG. 9. On the other hand, the image processing function F07 may also perform non-display processing on a non-display region including the feature 115 depicted in the X-ray image. This case will be described using FIG. 13.

FIG. 13 is a diagram showing display processing as an alternative to image processing, showing the feature 115 depicted in an X-ray image IM. The image processing function F07 sets a non-display region A in the X-ray image IM so that it includes the feature 115. The image processing function F07 performs non-display processing that can visually hide elements in the non-display region A, which is part of the X-ray image IM. For example, the image processing function F07 performs non-display processing of the elements in the non-display region A of the X-ray image IM using CSS (Cascading Style Sheets). This allows the elements in the non-display region A to be non-display when the X-ray image IM is displayed by the control device 20 of the X-ray diagnostic apparatus 1 or an external device (e.g., a viewer) of the X-ray diagnostic apparatus 1, and also allows the operator of the control device 20 or the viewer to selectively switch the elements in the non-display region A to be displayed.

In this way, the image processing shown in the divisions 9a to 9d of the FIG. 9 does not have to be performed to hide the features 115 of the X-ray image IM, and the image processing can be replaced with non-display processing. In this case, the processing time can be reduced compared to the case of image processing. In principle, all data from the X-ray exposure region is displayed. Since the non-display region A is very small and the image itself within the non-display region A is not processed, it is acceptable to hide the non-display region A.

According to at least one of the embodiments described above, it is possible to acquire, with a simple configuration, information indicating the relative positional relationship between the X-ray tube and the X-ray detector (e.g., SID information and oblique incidence angle information) that is required in the X-ray diagnostic apparatus.

While certain embodiments have been described, these embodiments have been presented by way of example only, and are not intended to limit the scope of the inventions. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. An X-ray diagnostic apparatus comprising:
an X-ray tube;
an X-ray diaphragm assembly including an X-ray diaphragm defining an irradiation field of X-rays emitted from the X-ray tube and having a characteristic object;
an X-ray detector configured to detect the X-rays emitted from the X-ray tube and passed through a subject; and
processing circuitry configured to calculate information indicating a relative positional relationship between the X-ray tube and the X-ray detector based on an image of the characteristic object depicted in an X-ray image being based on transmission data acquired by the X-ray detector.

2. The X-ray diagnostic apparatus according to claim 1, wherein the processing circuitry is configured to calculate a source-to-image distance (SID) as the information indicating the relative positional relationship, the SID being a distance from a focal point of the X-rays to the X-ray detector.

3. The X-ray diagnostic apparatus according to claim 1, wherein the processing circuitry is configured to calculate the information indicating the relative positional relationship based on a dimension of the characteristic object provided in the X-ray diaphragm assembly depicted in the X-ray image.

4. The X-ray diagnostic apparatus according to claim 1, wherein the processing circuitry is configured to calculate a ratio between a reference detection dimension and a dimension of the characteristic object depicted in an X-ray image acquired during the examination, and calculate an unknown SID using the known SID, the reference detection dimension being a dimension of the characteristic object depicted in the X-ray image acquired during a calibration scan with the SID set to the known SID.

5. The X-ray diagnostic apparatus according to claim 1, wherein the processing circuitry is configured to calculate the SID based on a ratio of the actual size of the characteristic object to the size depicted in the X-ray image and a distance of the characteristic object from the X-ray focal point.

6. The X-ray diagnostic apparatus according to claim 4, wherein
the characteristic object includes multiple members each having a characteristic shape provided on a side surface of at least one of multiple diaphragm blades constituting the X-ray diaphragm, and
the dimension of the characteristic object depicted in the X-ray image is a distance between the multiple members.

7. The X-ray diagnostic apparatus according to claim 6, wherein the characteristic shape includes at least one of a convex shape and a concave shape.

8. The X-ray diagnostic apparatus according to claim 4, wherein
the characteristic object includes single member having a characteristic shape provided on a side surface of at least one of multiple diaphragm blades constituting the X-ray diaphragm, and
the dimension of the characteristic object depicted in the X-ray image is a length of the single member.

9. The X-ray diagnostic apparatus according to claim 8, wherein the characteristic shape includes at least one of a convex shape and a concave shape.

10. The X-ray diagnostic apparatus according to claim 6, wherein
the characteristic object includes a convex portion provided on a first diaphragm blade being one of multiple diaphragm blades constituting the X-ray diaphragm, and a concave portion provided on a second diaphragm blade facing the first diaphragm blade, and
the convex portion and the concave portion are formed to fit together when the X-ray diaphragm is closed.

11. The X-ray diagnostic apparatus according to claim 8, wherein
the characteristic object includes a convex portion provided on a first diaphragm blade being one of multiple diaphragm blades constituting the X-ray diaphragm, and a concave portion provided on a second diaphragm blade facing the first diaphragm blade, and
the convex portion and the concave portion are formed to fit together when the X-ray diaphragm is closed.

12. The X-ray diagnostic apparatus according to claim 4, wherein the characteristic object is provided on a cover member being part of a case housing the X-ray diaphragm, the cover member being provided on a transmission window transmitting the X-rays toward the subject.

13. The X-ray diagnostic apparatus according to claim 12, wherein the characteristic object is a grid pattern provided on the cover member.

14. The X-ray diagnostic apparatus according to claim 1, wherein the processing circuitry is configured to calculate an oblique incidence angle of the X-rays with respect to the X-ray detector as the information indicating the relative positional relationship based on a degree of distortion of a grid pattern detected by the X-ray detector.

15. The X-ray diagnostic apparatus according to claim 1, wherein the processing circuitry is configured to perform delete processing to make the partial image corresponding to the characteristic object depicted in the X-ray image less noticeable.

16. The X-ray diagnostic apparatus according to claim 1, wherein the processing circuitry is configured to apply non-display processing to a region including the characteristic object depicted on the X-ray image.

17. The X-ray diagnostic apparatus according to claim 1, wherein
the characteristic object provided in the X-ray diaphragm assembly is part of a case housing the X-ray diaphragm, and includes a grid pattern provided on a cover member provided on a transmission window transmitting the X-rays toward the subject, and
the processing circuitry is configured to calculate an oblique incidence angle of the X-rays with respect to the X-ray detector as the information indicating the relative positional relationship from a degree of distortion of the grid pattern detected by the X-ray detector.

18. The X-ray diagnostic apparatus according to claim 1, wherein
the characteristic object provided in the X-ray diaphragm assembly includes a characteristic pattern containing positional information for at least three spatially separated points, and
the processing circuitry is configured to calculate an oblique incidence angle of the X-rays with respect to the X-ray detector as the information indicating the relative positional relationship based on a difference in shape between a first characteristic pattern provided in the X-ray diaphragm assembly and a second characteristic pattern detected by the X-ray detector.

19. The X-ray diagnostic apparatus according to claim 4, wherein the characteristic object includes a portion of a blade being all or part of multiple blades that constitute the X-ray diaphragm, a through hole for passing the X-rays being provided.
